(19) United States
(12) Patent Application Publication
(10) Pub. No.: US 2005/0209451 A1
(43) Pub. Date: Sep. 22, 2005

(54) Crystalline form of cefdinir

(76) Inventors: Antonio Manca, Milano (IT); Bruno Sala, Trezzo Sull'Adda (MI) (IT); Riccardo Monguzzi, Dorio (LC) (IT)

Correspondence Address: MANELLI DENISON & SELTER, 2000 M STREET NW SUITE 700, WASHINGTON, DC, 20036-3307, US

(21) Appl. No.: 11/129,425

(22) Filed: May 16, 2005

Related U.S. Application Data
(63) Continuation of application No. 10/405,648, filed on Apr. 3, 2003.

(30) Foreign Application Priority Data
Apr. 29, 2002 (IT) MI2002A 000913

(51) Int. Cl. A61K 31/545; C07D 501/14

(52) U.S. Cl. 540/222

(57) ABSTRACT

A new crystalline form of cefdinir having a dissolution rate less than that of the known crystalline form of the same product.

### BACKGROUND OF THE INVENTION

The present invention relates to a new crystalline form of cefdinir.

Cefdinir is a cephalosporin possessing high antibiotic activity and is described in U.S. Pat. Nos. 4,559,334 and 4,585,860.

U.S. Pat. No. 4,935,507 claims a crystalline form A of cefdinir, obtainable by crystallization at ambient temperature or by heating up to 40° C., within a pH range from 1 to 4, from a cefdinir solution prepared in accordance with the teachings of the two aforesaid patents.

### SUMMARY OF THE INVENTION

The present invention concerns a new crystalline form of cefdinir obtainable, at a temperature between 0° C. and +6° C., from a dilute aqueous solution of cefdinir in the presence of at least one organic solvent, in a total percentage (v/v on the aqueous solution) not exceeding 10% and at a pH between 1.5 and 3.

The product obtained in this manner has high stability and a dissolution rate less than that of crystal A, however it enables a final dissolved cefdinir value to be achieved which is higher than that possible with crystal A. These characteristics can be very advantageous in clinical practice, by also enabling high concentrations to be obtained which are prolonged in time.

### DETAILED DESCRIPTION OF THE INVENTION

To clarify the understanding of the present invention, one embodiment is described hereinafter by way of non-limiting example.

### EXAMPLE 1

An aqueous solution containing 108.6 g of moist cefdinir (syn isomer obtained in accordance with U.S. Pat. No. 4,559,334) in 3000 ml of water at pH 5.5 is decolorized with carbon (10 g) by agitating for 30 minutes. It is filtered, washed with 200 ml water and diluted with ethyl acetate (300 ml). The solution is cooled to 0° C./+2° C. and rapidly corrected to pH 2 by adding 6 N HCl. It is maintained under agitation at 0° C./+2° C. for 2 hours, filtered and washed with deionized water. The product is dried under vacuum at 25° C.
Yield: 96 μg of yellow crystalline powder;
K.F.: 6.0%;
Titre: 949 ig/mg on anhydrous base;
Total impurities: 0.10%.

Results superimposable on the aforeindicated were obtained operating with the same crystallization method, but using tetrahydrofuran (250 ml) instead of ethyl acetate and operating at a temperature of +5° C. The organic solvent can be formed from a mixture of organic solvents; the solution pH can be between 1.5 and 3; and the temperature can be between 0° C. and +6° C.

The X-ray diffraction spectrum of the new crystalline product obtained in accordance with the aforedetailed example is as follows:

| Anticathode: Cu Kα | Filter: Ni |
|---|---|
| Voltage: 40 kV | Current: 40 mA |
| d(A°) | Relative intensity |
| 15.24 | 30 |
| 11.30 | 18 |
| 10.92 | 18 |
| 7.51 | 100 |
| 5.66 | 24 |
| 5.48 | 55 |
| 4.91 | 20 |
| 4.76 | 96 |
| 4.55 | 44 |
| 4.23 | 71 |
| 4.17 | 85 |
| 3.99 | 74 |
| 3.74 | 18 |
| 3.64 | 78 |
| 3.53 | 24 |
| 3.46 | 62 |
| 3.39 | 85 |
| 3.26 | 14 |
| 3.17 | 21 |
| 3.08 | 37 |
| 2.96 | 10 |
| 2.89 | 23 |
| 2.82 | 69 |
| 2.81 | 42 |
| 2.63 | 13 |
| 2.57 | 21 |
| 2.54 | 18 |
| 2.39 | 8 |
| 2.31 | 17 |
| 1.99 | 25 |
| 1.97 | 10 |

This spectrum is shown in the accompanying figure.

Cefdinir in the new crystalline form was subjected to accelerated stability tests in comparison with crystal A claimed in U.S. Pat. No. 4,935,507. From these tests it emerged that the stability of the new crystal is at least equal to that of the reference crystal A. Dissolution tests in 0.07 N HCl were also effected, from which it emerged that the dissolution rate of the new crystal is less than that of crystal A. On prolonging the duration of the dissolution treatment it was found however that after the first hour the situation changed, in the sense that cefdinir in the new crystalline form was then more soluble than crystal A.

The organic solvents added to the aqueous cefdinir solution at the time of crystalization are preferably chosen from the group consisting of ethyl acetate and tetrahydrofuran.

1-3. (canceled)

4. A method for obtaining a crystalline form of cefdinir comprising:
adding at least one organic solvent in a percentage v/v up to 10% to an aqueous solution of cefdinir;
cooling the solution to a temperature between 0° C. and +6° C.; and
rapidly lowering the pH to between 1.5 and 3 to cause precipitation of the crystalline form of cefdinir having an X-ray diffraction spectrum of the following characteristics:

| Anticathode: Cu Kα | Filter: Ni |
|---|---|
| Voltage: 40 kV | Current: 40 mA |
| d(A°) | Relative intensity |
| 15.24 | 30 |
| 11.30 | 18 |
| 10.92 | 18 |
| 7.51 | 100 |
| 5.66 | 24 |
| 5.48 | 55 |
| 4.91 | 20 |
| 4.76 | 96 |
| 4.55 | 44 |
| 4.23 | 71 |
| 4.17 | 85 |
| 3.99 | 74 |
| 3.74 | 18 |
| 3.64 | 78 |
| 3.53 | 24 |
| 3.46 | 62 |
| 3.39 | 85 |
| 3.26 | 14 |
| 3.17 | 21 |
| 3.08 | 37 |
| 2.96 | 10 |
| 2.89 | 23 |
| 2.82 | 69 |
| 2.81 | 42 |
| 2.63 | 13 |
| 2.57 | 21 |
| 2.54 | 18 |
| 2.39 | 8 |
| 2.31 | 17 |
| 1.99 | 25 |
| 1.97 | 10. |

5. The method for obtaining the crystalline form of cefdinir according to claim 4, further comprising isolating the cefdinir from solution.

6. The method obtaining the crystalline form of cefdinir according to claim 4, wherein said organic solvent is chosen from the group consisting of ethyl acetate and tetrahydrofuran, used individually or mixed together.
